(19) United States
(12) Patent Application Publication
(10) Pub. No.: US 2013/0006103 A1
(43) Pub. Date: Jan. 3, 2013

(54) Micro-Devices for Biomedical Applications and Method of Use of Same

(75) Inventors: Chris C. Yu, Conneautville, PA (US); He Yu, Cheshire, CT (US)

(73) Assignee: Anpac Bio-Medical Science Co., LTD., Tortola (VG)

(21) Appl. No.: 13/555,858

(22) Filed: Jul. 23, 2012

Related U.S. Application Data
(62) Division of application No. 12/416,280, filed on Apr. 1, 2009.

(51) Int. Cl. A61B 17/22; A61B 6/12; A61B 10/02; A61M 5/00; A61B 18/20; A61B 17/00; A61B 18/02; A61B 18/18; A61B 18/12; A61B 5/00; A61M 37/00

(52) U.S. Cl. 600/424; 600/300; 600/562; 604/93.01; 604/20; 604/500; 606/1; 606/21; 606/14; 606/41; 606/159

(57) ABSTRACT

Among others, the present invention provides fabricated micro-devices for application in live biological systems, each comprising (a) an outer membrane, (b) a micro-mechanical, micro-chemical, micro-chemical-mechanical, micro-optical, micro-acoustical, micro-biological, micro-bio-chemical, micro-bio-chemical-mechanical, micro-electro-bio-chemical-mechanical, micro-electro-chemical-mechanical, micro-electro-bio-chemical-mechanical, micro-electro-mechanical, micro-electromagnetic-mechanical, micro-acoustic-mechanical, and micro-superconducting-mechanical properties, and (c) having a size as defined by the outer membrane ranging from 1 angstrom to 5 millimeters; and methods of using such fabricated micro-devices.

### CROSS-REFERENCE TO RELATED APPLICATIONS

Not Applicable

### FEDERALLY SPONSORED RESEARCH

Not Applicable

### BACKGROUND

The conventional approach to modern medicine, including prevention, diagnosis and treatment, has been mainly focused on macroscopic methodologies. For example, current diagnosis of disease techniques use macroscopic data and information such as temperature, blood pressure, scanned images, measured chemical component levels in the body, etc. Even the effectiveness of newly-emerged DNA tests in diagnosing a wide range of diseases in a real-time, reliable, accurate, rapid, and cost efficient manner has not been established. Many diseases with great morbidity and mortality, including cancer and heart disease, are very difficult to diagnose early and accurately. Further, most of the existing diagnosis techniques are invasive.

Relating to disease treatment, the situation is even worse. To date, many operations are still highly invasive, have a high cost, contain a high risk of complications and require a long recuperation time. Some treatments are even destructive of healthy cells and/or tissue. One such example would be cancer treatment using radiation, which not only kills cancer cells; it also kills normal, healthy cells. Yet another example would be blood related disease treatment which is often intrusive, risky (e.g. open heart surgery), highly expensive and in many cases, post-surgical patients will not be able to return to a normal active life style.

On the prevention side of the equation, beside the general guidelines of eating healthy and exercising regularly, the cause of many diseases, such as cancer, are still unknown at this point. This lack of knowledge relating to disease etiologies directly leads to a lack of preventative drug development.

Most of the above stated issues in prevention, diagnosis, and treatment in modern medicine are, to a large extent, due to the following:
lack of understanding of pathology at the microscopic level (cell biology level),
lack of effective drug delivery and efficient reaction mechanisms,
lack of non-invasive monitoring at the microscopic level as well as preventive mechanisms and approaches, and
lack of non-invasive, effective, targeted disease treatment approaches and technologies.

In recent years, there have been some efforts in the areas of using nano-technologies for biological applications, mostly for use in vitro (outside the body). This in vitro work has lead to moderate developments in the field. Pantel, et al., discussed the use of a micro-electromechanical (MEMS) sensor for detecting cancer cells in blood and bone marrow in vitro [See Klaus Pantel, et al., “Detection, Clinical Relevance and Specific Biological Properties of Disseminating Tumor Cells”, p. 329, vol. 8, Nature Reviews, (2008).]. Wozniak and Chen used laser tweezers and micro-needles for measuring forces generated by sample cells (also in vitro) [See M. A. Wozniak and C. S. Chen, “Mechanotransduction in Development: a Growing Role for Contractility”, p. 34, vol. 10, Nature Reviews (2009).]. Kubena et al., disclosed, in U.S. Pat. No. 6,922,118, the deployment of MEMS for detecting biological agents, while Weissman et al., conceived the idea, in U.S. Pat. No. 6,330,885, of utilizing MEMS sensor for detecting accretion of biological matter.

However, to date, most of the prior art has been limited to isolated examples for sensing in vitro, using systems of relatively simple constructions and large dimensions and often with limited functions. There is no prior art in the area of highly integrated, multi-functional, micro-devices (less than or equal to 5 millimeters) for advanced biomedical applications, particularly for applications in vivo (inside the body) and at the microscopic level. Due to the above stated limitations, at the fundamental level, many issues facing modern medicine remain unsolved, including sensing at the microscopic level in vivo targeted treatments, cancer prevention, early detection and non-invasive treatment with minimum damage to normal tissues and organs.

### SUMMARY

The present invention is directed to the use of novel micro-devices for carrying out disease prevention, diagnosis, and treatment at microscopic levels, using a wide range of novel functions achieved through their functionality integration at the microscopic level and using the state-of-the-art micro-device fabrication techniques such as integrated circuit fabrication techniques.

Such fabrication techniques include, but are not limited to, mechanical, chemical, chemical-mechanical, electro-chemical-mechanical, electro-bio-chemical- mechanical, integrated circuit and semiconductor manufacturing techniques and processes. Depending upon its application, the micro-device size in the present invention can range from 1 angstrom to 5 millimeters. Micro-device functionalities would at least include sensing, detecting, measuring, diagnosing, monitoring, analyzing, drug delivering, selective absorption, selective adsorption, carrying out preventive procedures and surgical intervention.

The term “micro-device” as used in the present application has a general meaning for an application from a single material to a very complex device comprising of multiple materials with multiple sub-units with multiple functions. The micro-device in the present invention can range from about 1 angstrom to about 5 millimeters, with a preferred size from about 1 angstrom to 100 microns for devices targeted at biological systems of small size such as cell structures, DNA, and bacteria applications and a preferred size from about 0.01 micron to about 5 millimeters when targeting relatively large biological matters such as a portion of a human organ. As an example, a simple micro-device defined in the present application can be a single particle of a diameter less than 100 angstroms, with desired surface properties (such as surface charge or a coated chemical composition) for preferential absorption or adsorption into a targeted type of cell. The word “absorption” typically means a physical bonding between the surface and the material attached to it (absorbed onto it, in this case). On the other hand, the word “adsorption” generally means a stronger, chemical bonding between the two. These properties are very important for the present invention as they can be effectively used for targeted attachment by desired micro-devices for (a) measurement at the microscopic level, (b) targeted removal of unhealthy cells, and (c) protection of healthy cells during a treatment such as laser surgery.

Through novel micro-devices, their novel combinations and integrations, and integrated operating process flow, many issues in today's medicine can be solved. In particular, with the present invention, a micro-device can be used in “cleaning” biological organs including cleaning veins to prevent heart attack, strokes and blood clogging due to plaques and fatty deposits in the veins. Another innovative aspect of the present invention is the use of micro-devices for obtaining real time data and information at the cell structure level in a non-invasive manner, such as using a micro-voltage comparator, four-point probe and other circuitry designs to measure cell surface charge. The cell surface charge differentiation can be an important factor in deciding the healthy or unhealthy status of a cell and, accordingly, the proper treatment thereof. One example would be the use of such devices for measuring surface and/or bulk electrical properties including resting potential and surface charge for differentiating normal cells and cancer cells.

Yet another aspect of the present invention is the use of a micro-device to deliver drugs to targeted locations within the human body and with differentiation between healthy cells and unhealthy (cancer, for instance) cells. This can be achieved through selective absorption or adsorption of a micro-device onto healthy or unhealthy cells (such as cancer cells). For example, to remove a part of an unhealthy organ with laser surgery, micro-devices with high optical reflectivity can be used to selectively adsorb onto healthy cells, thereby protecting good cells from being removed and/or ablated via laser treatment.

### BRIEF DESCRIPTION OF THE DRAWINGS

These and other features, aspects and advantages of the present invention will become better understood with regard to the following description, appended claims, and accompanying drawings where:

FIG. 1 illustrates a perspective view of a micro-device that can act as a micro-injector showing the micro-device before and then after the injection process has completed.

FIG. 2 illustrates a perspective view of a micro-device that acts as a micro-polisher.

FIG. 3 illustrates a perspective view of a micro-device that acts as a micro-polisher, a micro-filter, a micro-injector, a micro-sensor and micro-shredder.

FIG. 4 illustrates a perspective view of a micro-device that acts as a micro-knife.

FIG. 5 illustrates a perspective view of a micro-device that acts as a micro-filter.

FIG. 6 illustrates a perspective view of a micro-device that acts as a micro-shield.

FIG. 7 illustrates a perspective view of a micro-device in a blood vessel as it nears a plaque in the vessel wall.

FIG. 8 illustrates a perspective view of a micro-device in a blood vessel as it senses a change in pressure around a plaque, triggering the micro-device's cleaning function.

FIG. 9 illustrates a perspective view of a micro-device in a blood vessel after said device has cleaned a plaque from the vessel wall.

FIG. 10 illustrates a perspective close up view of a group of healthy cells and a group of unhealthy, cancerous cells.

FIG. 11 illustrates a perspective close up view of a group of healthy cells and a group of unhealthy, cancerous cells with micro-devices acting as a voltage comparator on both sets of cells.

FIG. 12 illustrates a perspective close up view of a group of healthy cells and a group of unhealthy, cancerous cells.

FIG. 13 illustrates a perspective close up view of a group of healthy cells and a group of unhealthy, cancerous cells with micro-devices either adsorbed or absorbed onto the healthy cells only.

FIG. 14 illustrates a perspective close up view of an integrated micro-device with various sub-units comprising of a micro-cutter, a micro-needle, a memory unit, a unit for analysis and logic processing, a micro-sensor and a signal transmitter.

FIG. 15 illustrates a perspective view of a micro-device with a sensing unit, logic unit and micro-injector.

### DESCRIPTION

The present invention is directed to novel micro-devices for biological applications, which are expected to resolve a number of critical issues in the modern approach to medicine. These issues include the lack of understanding in pathology and prevention for a number of deadly diseases, lack of non-invasive, microscopic and effective diagnosis of various disease states, and a lack of an effective and targeted drug delivery system and treatment for deadly diseases such as cancer.

The micro-device disclosed in the present invention is a device ranging in size from about 1 angstrom to about 5 millimeters. In general, a smaller micro-device size is the preferred embodiment for sensing, measuring, and diagnostic purposes, particularly for obtaining information and data at the cell structure and DNA levels, where the preferred micro-device size is from about 1 angstrom to about 100 microns. When surgical operations will utilize a micro-device on a part of a human organ of larger size, a relatively large micro-device size is the preferred embodiment (100 microns to 5 millimeters in size), with the exception of manipulation at the cell structure level.

As stated herein, the general term “micro-device” can mean a wide range of materials, properties, shapes, and degree of complexity and integration. The complexity contemplated in the present invention ranges from a very small, single particle with a set of desired properties to a fairly complicated, integrated unit with various functional units contained therein. For example, a simple micro-device could be a single spherical article of manufacture of a diameter as small as 100 angstroms with a desired hardness, a desired surface charge, or a desired organic chemistry absorbed on its surface. A more complex micro-device could be a 1 millimeter device with a sensor, a simple calculator, a memory unit, a logic unit, and a cutter all integrated onto it. In the former case, the particle can be formed via a fumed or colloidal precipitation process, while the device with various components integrated onto it can be fabricated using various integrated circuit manufacturing processes.

The micro-devices of the present invention have a wide range of designs, structures and functionalities. They include but are not limited to a voltage comparator, a four-point probe, a calculator, a logic circuitry, a memory unit, a micro-cutter, a micro-hammer, a micro-shield, a micro-dye, a micro-pin, a micro-knife, a micro-needle, a micro-thread holder, micro-tweezers, a micro-optical absorber, a micro-mirror, a micro-wheeler, a micro-filter, a micro-chopper, a micro-shredder, micro-pumps, a micro-absorber, a micro-signal detector, a micro-driller, a micro-sucker, a micro-tester, a micro-container, a signal transmitter, a signal generator, a friction sensor, an electrical charge sensor, a temperature sensor, a hardness detector, an acoustic wave generator, an optical wave generator, a heat generator, a micro-refrigerator and a charge generator.

As disclosed herein, the range of functionality and applications using the said micro-devices can be made extremely powerful due to their diverse properties, high degree of flexibilities, and ability of integration and miniaturization.

Further, it should be noted that advancements in manufacturing technologies have now made fabrications of a wide range of micro-devices and integration of various functions onto the same device highly feasible and cost effective. The typical human cell size is about 10 microns. Using the state-of-the-art integrated circuit fabrication techniques, the minimum feature size defined on a micro-device can be as small as 0.1 micron. Thus, it is ideal to utilize the disclosed micro-devices for biological applications.

In terms of materials for the micro-devices, the general principle will be a material's compatibility with biological materials. Since the time in contact with a biological cell or group of cells may vary, depending on its applications, different materials may be selected. In some special cases, the materials may dissolve in a given pH in a controlled manner and thus may be selected as an appropriate material. Other considerations include cost, simplicity, ease of use and practicality. With the significant advancements in micro-fabrication technologies such as integrated circuit manufacturing technology, highly integrated devices with minimum feature size as small as 0.1 micron can now be made cost effectively and commercially. One good example is the design and fabrication of micro-electro-mechanical devices (MEMS), which now are being used in a wide variety of applications in the integrated circuit industry.

The following sections include several examples of the use of various novel types of the present micro-device invention for novel biological applications.

### Sensing, Measuring, and Diagnosis

Until the invention disclosed herein, there has been no probe to measure microscopic properties, in real time, at the cellular level in living organs (in vivo). A novel micro-device is disclosed herein, which measures cell properties in living organs. Further, it is expected that the measured information can be retrieved in real time for use as a diagnostic tool.

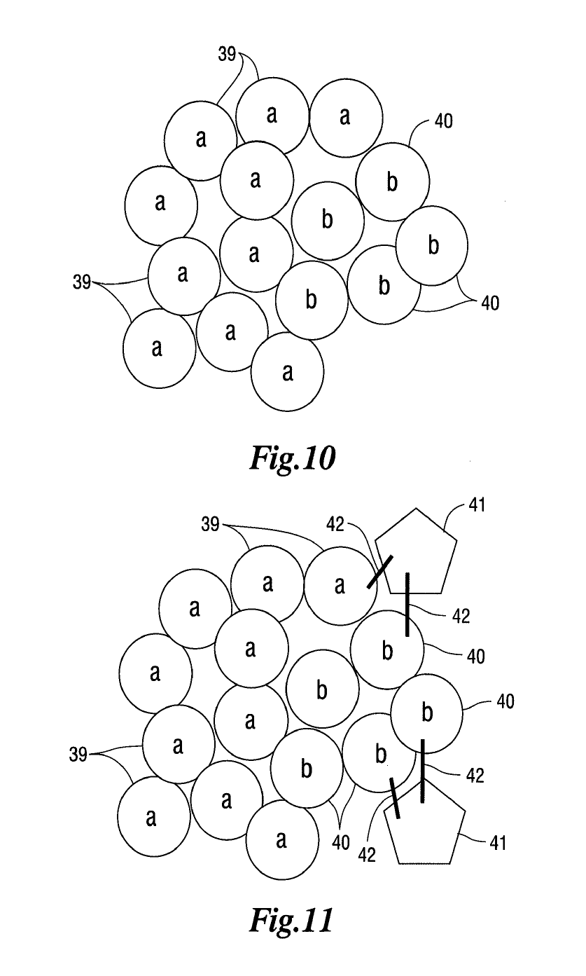

For example, a micro-device can be utilized to detect a cancer cell in a living organ in a non-invasive manner. FIG. 10 illustrates an area in the human body with a number of healthy cells “a” 39 and a number of unhealthy cells “b” 40. The electrical properties such as electrical charge and resting potential on healthy cells “a” 39 are different than the electrical properties on unhealthy cells “b” 40. First, the micro-device with a voltage comparator is calibrated by measuring surface charge (or voltage) at known healthy cells. Next, as shown in FIG. 11, for an area containing both healthy (or normal) cells 39 and unhealthy (or abnormal) cells 40, a micro-device 41 with voltage comparators 42 is used to scan the area. By comparing voltages at the cell surface (the difference in charges and/or potential), unhealthy cells 40 can readily be differentiated from the healthy cells 39. Such micro-devices 41 can be easily extended to perform both measuring and treating of cancer cell functions by integrating a voltage comparator, a logic circuitry unit, and a micro-injector (needle), which can deliver, for example, cancer-killing agents specifically to a cancer cell.

### Drug Delivery

To date, many cancer treatment drugs have not shown their expected promising results in human trials, even though laboratory tests on mice may have been successful. The inventors of this application believe that there may be major problems relating to the successful and effective drug delivery to the targeted cancer cells. Since such drugs are often taken in pill form or by injection into the body, there may be serious issues in the drug reaching the targeted cancer sites. Even if it can reach its targeted site, a drug's strength (concentration) and chemical composition may have been altered, rendering it either partially or entirely ineffective. An increase in the amount of drug delivered in this fashion will increase side effects and possibly cause an increase in mortality.

In the present invention, the novel, effective and targeted drug delivery system hopes to correct the above stated problems. As shown in FIG. 15, a micro-device 64 with a sensing unit 62, a logic unit 63 and a micro-injector 61 is utilized. The micro-device 64 is designed in a way that it will preferentially absorb (or adsorb) only onto unhealthy cells. Alternatively, the said sensor 62 can detect unhealthy cells through measurements of desired physical, chemical, electrical and biological properties of cells being scanned and attached onto detected unhealthy cells. Once the micro-device 64 is attached to the unhealthy cell, it will inject cancer-killing agent(s) into the cancer cell through a micro-injector 61. To make sure that healthy cells are not injected due to error in attachment, a logic unit 63 may be used to make a correct decision based on the sensor data received by the sensing unit 62 from the attached cell. Since this approach is a targeted approach with a cancer-killing drug directly delivered to the unhealthy cells, it is expected that its effectiveness can be greatly improved over the standard therapies that are used conventionally for the current treatment of cancer.

### Cleaning

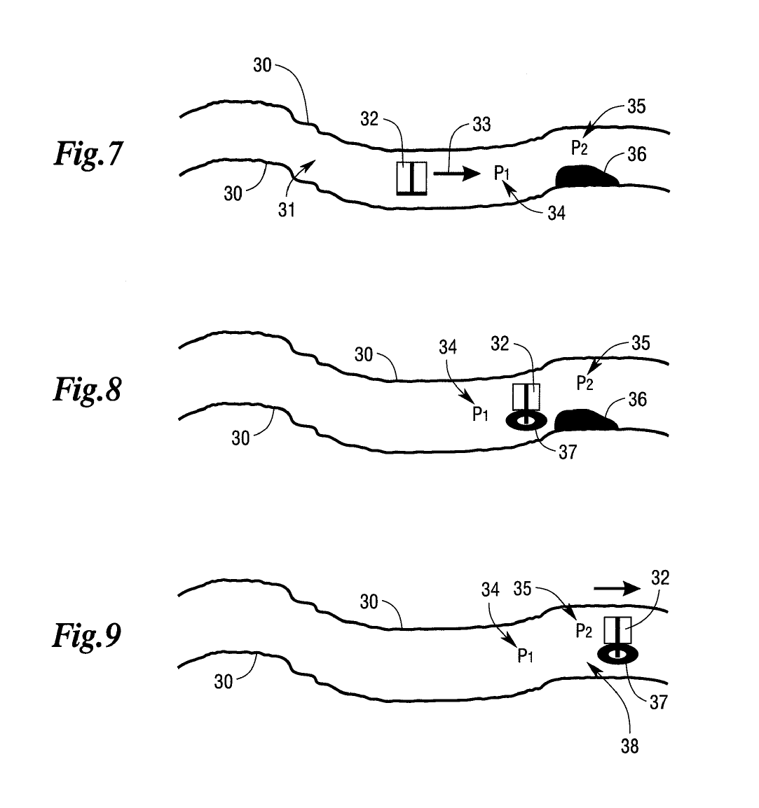

Another major area of focus for this invention is a novel type of micro-device for biological “cleaning” purposes. In particular, for the “cleaning” of human arteries and veins. FIG. 7 illustrates a blood vessel wall 30, a micro-device 32 traveling in a direction 33, a blood clot 36, lower blood pressure P1 34 and a lower blood pressure P2 35. In this type of application, the present invention is a micro-device 32 with at least one cleaner attached thereto. A more complete micro-device will be comprised of at least one sensor, one cleaner, one micro-filter, one-injector, one shredder and one pump. As shown in FIG. 8, a micro-device 32 with integrated functions of sensing (for local pressure measurement) and cleaning 37 can be used for arteries and vein cleaning applications. In this case, local pressure is higher where a plaque 36 is located at P2 35 within the blood vessel wall 30. The device is moving within the vessel walls 30 in direction 33 toward the plaque 36. The device 32 senses this increase in local pressure as it approaches the plaque, triggering the cleaning function 37 to be deployed. FIG. 9 illustrates the blood vessel wall 30 after the micro-device 32 with cleaning function 37 has cleaned the plaque from an area 38 within said blood vessel wall 30. This is just one of the many examples where a micro-device disclosed in this application can be used as a “smart” device for biological applications in a non-invasive, real time manner.

In FIG. 3, a more refined micro-device 15 is disclosed, which is comprised of cleaner arms 8 and cleaners 9, sensors 15, micro-filters 13 and 14, micro-shredders 11, and micro-injectors 16. This design is aimed to (a) facilitate the cleaning process and (b) make sure that cleaning debris is reduced to much smaller pieces so that it is completely removed and will not cause a clot in other areas of the human body. The cleaner typically has a polishing or rubbing capability, while filters are used to filter debris from cleaning and prevent them from moving to other parts of the body and cause clogging problems. The injector is used to dispense a dissolution agent to dissolve the debris from the cleaner portion of the micro-device; it can also deliver agent(s) to facilitate the “cleaning” (polishing) process. A micro-shredder 11 can be used to shred the relatively large debris from the cleaning (if any) activity. More specifically, the cleaning unit can be a polishing pad 9 made of polymer material(s) with desired roughness for polishing or rubbing. To reduce mechanical force and avoid breakage of the plaque into large pieces, a polishing solution can be applied at the point of micro-polishing, with the use of an injector 16. In a preferred method, the plaque is polished off in a layer by layer (a few mono-layers of about 10 angstroms in thickness) process, with a controlled removal rate. A balanced chemical-mechanical polishing process is preferred where both surface chemical reaction and mechanical abrasion is present, with the mechanical abrasion controlled to a low enough level not to cause breakage in plaque. In the meantime, micro-filters 13 and 14 are used to insure that no large debris can leave the area of cleaning and causing damage to other portions of the human body. For patients with a propensity for deposits building up in their veins, cleaning using the disclosed method should be carried out on a regular basis to reduce the risks of heart attack and stroke, and to reduce the degree of difficulty in subsequent cleaning processes.

Since the diameter for major arteries is typically a few millimeters (about 2 mm to 4 mm in diameters), the size for a micro-device for this type of cleaning application (for cleaning of major arteries) is from about 10 microns to less than 2 millimeters, with a preferred size of from about 100 microns to about 1.5 millimeters.

### Targeted Treatment

The micro-devices disclosed in this invention are ideally suited for targeted medical treatment to remove or destroy unhealthy cells or organ portions while minimizing damage to the unhealthy cells or organ parts. This can be carried out with a high degree of selectivity, can be non-invasive and can be done in a microscopic manner.

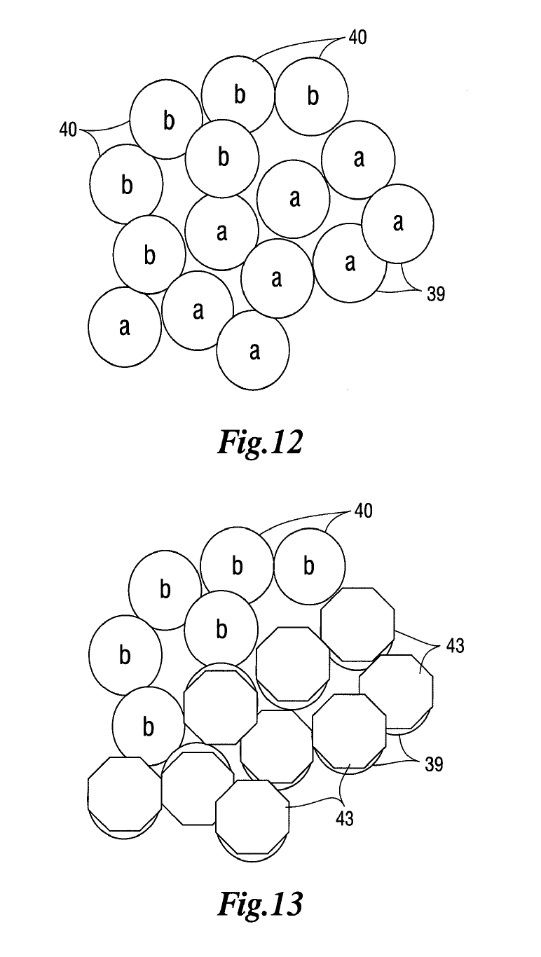

FIG. 12 illustrates an area in the human body with a number of healthy cells 39 and a number of unhealthy cells 40. In FIG. 13, for use in laser surgery using an optical oblation process, healthy cells 39 are first covered with micro-devices 43 (called micro-shields) with a high optical reflectivity. Next, unhealthy cells 40 such as cancer cells are removed via optical oblation, while healthy cells 39 are protected by the micro-shields 43. This selective attachment of the micro-shields 43 to healthy cells is made possible through surface adsorption (or absorption) between said micro-devices and healthy cells through micro-device sensing process and/or desired micro-device properties such as charge attraction. For example, micro-devices can be designed or programmed such that they only attach to healthy cells through surface charge measurement and subsequent logic decision and action as set forth in FIG. 11 described above.

Another preferred embodiment of the present invent to target treatment is the use of an integrated micro-device with sensing, logic processing, and injection functions. Said micro-device first uses a sensing function to locate its target. Said micro-device then attaches itself to the target. Finally, said micro-device injects cancer-killing agent(s) into the cancer cell.

### Micro-Surgery

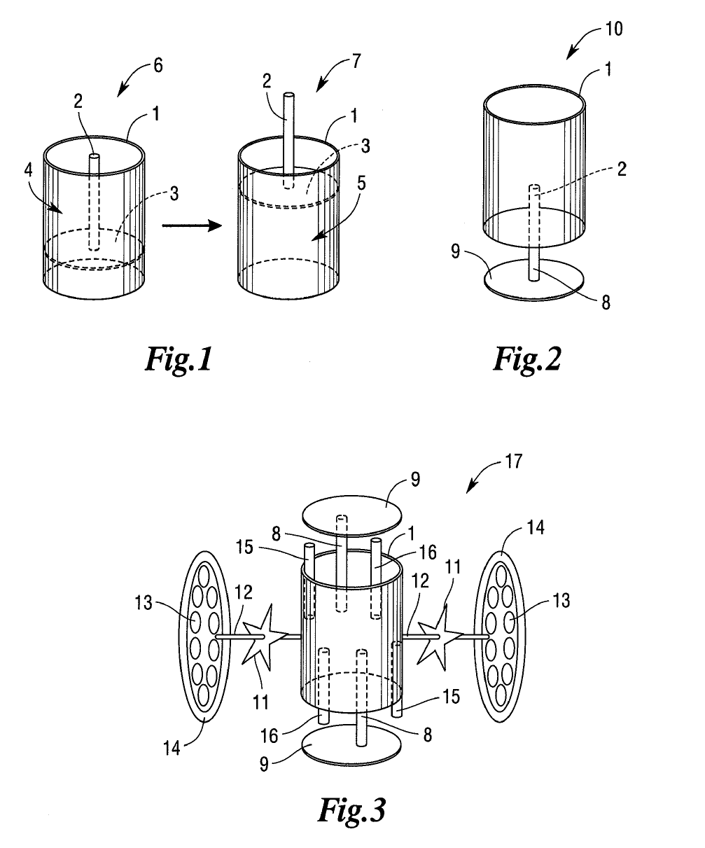

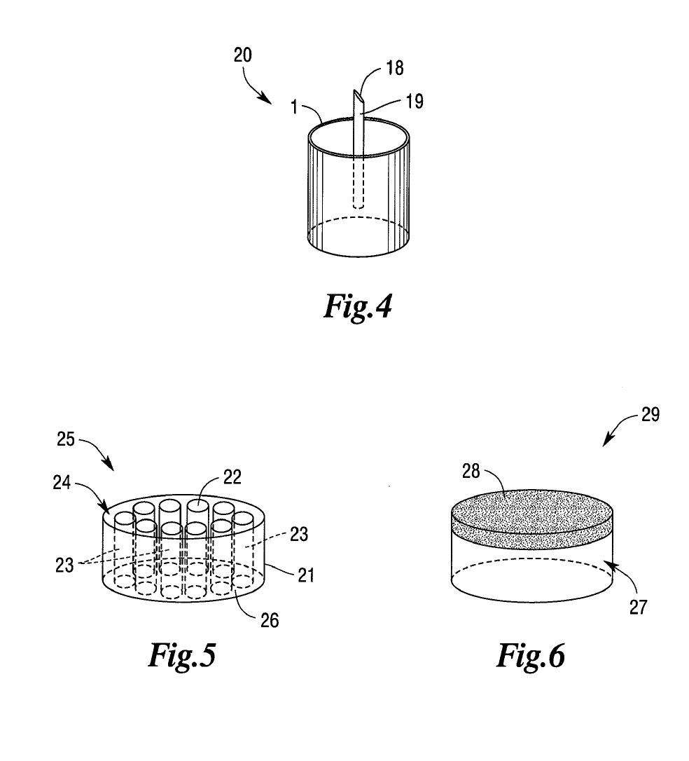

As disclosed herein, various micro-devices capable of performing a wide range of surgical functions can be employed to accomplish specific goals. Some examples of the said micro-devices capable of carrying out micro-surgeries are shown in FIGS. 1 through 6. FIG. 1 illustrates a micro-device 6 before it is triggered and a micro-device 7 after it is triggered. Said device 6 is comprised of an outer membrane 1, a sensing unit 2, a floor 3 and an area 4 in which various agents can be held prior to triggering. Said triggered device 7 has an area 5 which is empty once the floor 3 is pushed vertically to expel the contents of the area 4. FIG. 2 illustrates a micro-device 10 with a polisher/scrubber function 9 attached to an extension arm 8 outside of the outer membrane 1. FIG. 4 illustrates a micro-device 20 with an outer membrane 1, a vertical attachment 19 with a cutting knife end 18. FIG. 5 illustrates a micro-device 25 with a top side 24, an outer membrane 21, a series of openings 22 in said top side 24 with said openings 22 extending through passage 23 entirely through micro-device 25 to the bottom side 26. FIG. 6 illustrates a micro-device 29 having a body 27 with a reflective portion 28 attached to the top of said body 27.

It should be emphasized that for practical surgical applications, integrated micro-devices with multiple functional components and functionalities will be the preferred choices, and they will be the most effective and versatile instruments for surgeries. The clear advantages of those “smart” devices disclosed in this invention will be to carry out surgery in a minimally invasive and at a microscopic level with high precision, high selectivity, with minimum damage to healthy cells and organs.

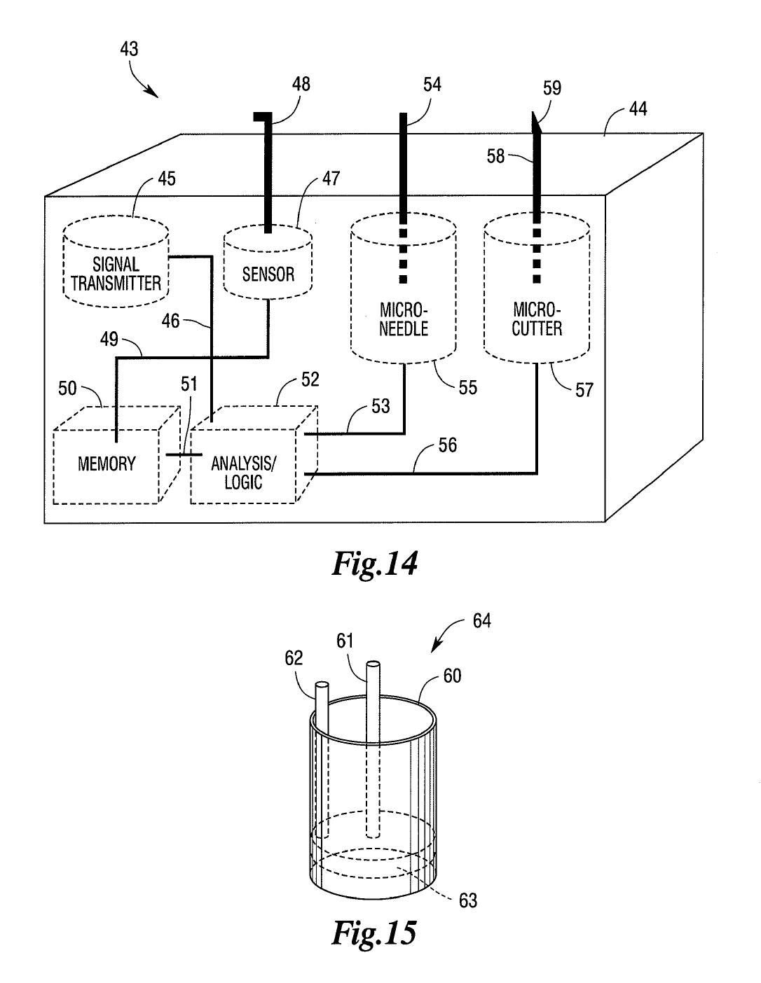

One preferred example is an integrated micro-device with at least one sensor, one memory unit, one logic processing unit, one signal transmitter, one signal receiver, at least one micro-injector, multiple micro-knives, multiple micro-needles, at least one pair of micro-tweezers, and at least one micro-thread holder. Such integrated micro-device will be capable of performing some basic surgical operations. One such example of integrated micro-devices is shown in FIG. 14. FIG. 14 illustrates an integrated micro-device 43 with an outer membrane 44, a sensing unit 47 attached to a sensing arm 48 linked to a memory unit 50 via pathway 49, said memory unit 50 linked via pathway 51 to an analysis/logic unit 52, said unit 52 attached via pathway 46 to a signal transmitter 45, said unit 52 attached via pathway 53 to a micro-needle unit 55 reaching externally via a needle 54 extending past said outer membrane 44 and said unit 52 attached via pathway 56 to a micro-cutter unit 57 with an extending arm 58 having a cutting end 59.

Thus it is apparent that there has been provided, in accordance with the invention disclosed herein, a micro-device for biological applications, particularly for disease detection, treatment, and prevention in live biological systems at a microscopic level, that fully meets the needs and advantages set forth herein. Although specific embodiments have been illustrated herein, it will be appreciated by those skilled in the art that any modifications and variations can be made without departing from the spirit of the invention. Therefore, it is not intended that the invention be limited to the said embodiments. Any combination of the micro-devices disclosed in this invention and any obvious extension of the said micro-devices for biological applications would be covered in the spirit of this invention. Additionally, any integration of disclosed micro-devices for disease detection, prevention and treatment including surgical operations in live human body disclosed herein. Therefore, it is intended that this invention encompass any arrangement, which is calculated to achieve that same purpose, and all such variations and modifications as fall within the scope of the appended claims.

The reader's attention is directed to all papers and documents which are filed concurrently with this specification and which are open to public inspection with this specification, and the contents of all such papers and documents are incorporated herein by reference. All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

Any element in a claim that does not explicitly state “means for” performing a specific function, or “step for” performing a specific function, is not to be interpreted as a “means” or “step” clause as specified in 35 U.S.C.§112 para. 6. In particular, the use of “step of” in the claims herein is not intended to invoke the provisions of 35 U.S.C.§112 para. 6.

1. A fabricated micro-device for application in live biological systems, said micro-device comprising
(a) an outer membrane; (b) at least one of the following properties selected from the group consisting of : micro-mechanical, micro-chemical, micro-chemical-mechanical, micro-optical, micro-acoustical, micro-biological, micro-bio-chemical, micro-bio-chemical-mechanical, micro-electro-bio-chemical-mechanical, micro-electro-chemical-mechanical, micro-electro-bio-chemical-mechanical, micro-electro-mechanical, micro-electromagnetic-mechanical, micro-acoustic-mechanical, and micro-superconducting-mechanical properties; and (c) having a size as defined by the outer membrane ranging from 1 angstrom to 5 millimeters.

3. A fabricated micro-device for application outside a biological system, said micro-device comprising (a) an outer membrane; (b) at least one of the following properties selected from the group consisting of: micro-mechanical, micro-chemical, micro-chemical-mechanical, micro-optical, micro-acoustical, micro-biological, micro-electro-mechanical, micro-electromagnetic-mechanical, micro-acoustic-mechanical, and micro-superconducting-mechanical properties; and (c) having a size ranging from 1 angstrom to 5 millimeters.

4. The fabricated micro-device in claim 1, wherein said micro-device has desired properties for preferential adsorption onto targeted biological organ and cell structure surfaces.

5. The fabricated micro-device in claim 1, wherein said micro-device has desired properties for preferential absorption onto targeted biological organ and cell structure surfaces.

6. The fabricated micro-device in claim 1, wherein said micro-device has means to differentiate cancer cells from normal cells.

8. The fabricated micro-device in claim 1, wherein said micro-device can perform at least one function selected from the group consisting of: measure microscopic properties of organ and cell structures, diagnose organ and cell structures at a microscopic level, deliver desired chemistry to organ and cell structures at a microscopic level, deliver desired drug to organ and cell structures at a microscopic level, and manipulate selected organ and cell structure at a microscopic level, in a non-invasive manner.

9. The fabricated micro-device in claim 1, wherein said micro-device can perform at least one function selected from the group consisting of: measure microscopic properties of organ and cell structures, diagnose organ and cell structures at a microscopic level, deliver desired chemistry to organ and cell structures at a microscopic level, deliver desired drug to organ and cell structures at a microscopic level, and manipulate selected organ and cell structure at a microscopic level, in real time.

10. The fabricated micro-device in claim 1, wherein said micro-device has the function to measure microscopic properties including at least one property selected from the group consisting of: surface charge, resting potential, electro-chemical potential, electrical potential, surface wettability, contact angle, adhesion, temperature, density, friction, hardness, surface tension, trace chemical concentration, hydrophobic level, hydrophilic level, pH, liquid flow rate, pressure, optical properties, absorption, adsorption, and composition.

11. The fabricated micro-device in claim 1, wherein said micro-device has hardware and means for local positioning, location identification, location information communication and location positioning.

13. The fabricated micro-device in claim 1, wherein said micro-device has a pre-programmed trigger function for actions selected from the group consisting of: chemistry delivery, mechanical force action, charge injection, light emitting, voltage application, cooling and heating onto organic structures.

14. The fabricated micro-device in claim 13, wherein said trigger function is achieved by the employment from a group of parameters selected from the group consisting of: charge, resting potential, electrical potential, electro-chemical potential, surface current, bulk current, surface wettability, adhesion property, hydrophobic level, hydrophilic level, flow property, electrical field, magnetic field, acoustic field, temperature, light wavelength and/or intensity, frictional force and coefficient, hardness, pressure and external signal detected by the device.

15. The micro-device in claim 1, wherein said micro-device optionally has a dissolution capability at a targeted pH range between 30 seconds and three (3) days.

19. The fabricated micro-device in claim 1, wherein said micro-device has a size from 1 angstrom to 100 microns for selective attachment applications.

20. The fabricated micro-device in claim 1, wherein said micro-device comprises at least one material with multiple sub-devices integrated onto one unit with at least one functionality.

23. The fabricated micro-device in claim 20, wherein said micro-device has a preferred size from 0.01 micron to 5 millimeters.

24. The fabricated micro-device in claim 1, wherein said micro-device is optionally an integrated micro-device comprising of at least one function selected from the group consisting of: detecting, measuring, calculating, analyzing, diagnosing, logic processing (decision making), transmitting, and operating/surgical hardware and functions.

27. The fabricated micro-device in claim 1, wherein said micro-device has means for cleaning comprising: cleaners, filters, shredders, injectors and pumps.

28. The fabricated micro-device in claim 1, wherein said micro-device has means for injecting a desired chemical component to the location to be cleaned comprising: at least one cleaner, at least one micro-filter, at least one shredder, and at least one injector.

29. The fabricated micro-device in claim 1, wherein said micro-device comprises at least a polishing unit with a polishing pad.

33. The fabricated micro-device in claim 1, wherein said micro-device is a micro-tester for continued scan and analysis of live biological system for early disease detection and prevention.

34. The fabricated micro-device in claim 33, wherein said micro-tester comprises: (a) sensors; (b) micro-tip for sample collection; (c) micro-arrays for testing collected sample; (d) data analysis unit; and (e) signal transmitter.

35. The fabricated micro-device in claim 34, wherein said micro-tester has a preferred size from 1 micron to 100 microns for human cell tests and analysis.

36. The fabricated micro-device in claim 34, wherein said micro-tester has a preferred size from 10 microns to 5 millimeters for human organ tests and analysis.

37. The fabricated micro-device in claim 34, wherein said micro-tester has a preferred size from 2 angstroms to 50 microns for tests and analysis of bacteria, human DNA, and cells of relatively small to medium sizes.

38. The fabricated micro-device in claim 37, wherein said micro-tester is employed for early cancer detection and prevention in vivo.

39. The method of using a fabricated micro-device whereby said micro-device is used for biological cleaning functions.

40. The method of using a fabricated micro-device in claim 39, wherein use of the said micro-device is for cleaning human veins and arteries for preventing heart attacks, strokes, and any form of blood clogging.

41. The method of using a fabricated micro-device in claim 40, comprising the steps of: (a) delivering a micro-device to the general area where cleaning is to be carried out; (b) optionally measuring local parameters selected from a group comprising: local temperatures, local pressures, local frictional forces, local surface charges, local resting potentials, local electrical potentials, local surface properties, local compositions and local fluid flow rates; (c) optionally triggering cleaning function; (d) performing cleaning (e) optionally collecting debris from cleaning by a micro-collector and transporting said debris away; and, (f) optionally collecting debris by a micro-filter and transporting said debris away.

42. The method of using a fabricated micro-device in claim 41, wherein a type of or combinations of types of micro-devices for cleaning plaques in human veins comprising the steps of: (a) delivering a micro-device into the veins; (b) optionally sensing and analyzing data being collected, for instance local pressure; (c) optionally triggering cleaning functions when the targeted blood vein location is reached; (d) cleaning plaque and deposits from the vein wall at the targeted location; (e) optionally, injecting desired chemistry into the blockage to be cleaned to soften the plaque being cleaned, avoid formation of large debris from breakage from plaque, and minimize possible damage to the veins; (f) optionally dissolving said micro-device following completion of cleaning or filtered out via blood filtration; (g) optionally filtering of said micro-device and debris via blood filtration during and following completion of cleaning; and, (h) optionally carrying out post-cleaning treatments by said micro-device.

43. The method of using a fabricated micro-device in claim 40, wherein said cleaning is carried out by at least one of the following means: mechanical polishing, mechanical rubbing, chemical-mechanical polishing, chemical dissolution, chemical passivation, chemical treatments, biological treatments, polishing with chemical dissolution, and laser oblation.

44. The method of using a fabricated micro-device for delivering multiple doses of drug comprising: (a) transportation of micro-device to the desired location in vivo; (b) delivering the first drug to the target; and (c) delivering a second dose of the drug to the same target within a desired time interval from the delivery time of the initial delivery.

45. The method of using a fabricated micro-device in claim 44, wherein the multiple doses are comprised of different drugs.

46. The method of using a fabricated micro-device in claim 44, wherein the drugs are of different chemistries and delivering of a first drug will enhance the attachment selectivity to the second drug.

47. The method of using a fabricated micro-device, wherein one type or a combination of at least two types of micro-devices perform activities selected from the group consisting of: drug delivery, cutting, removing, polishing, transporting, jointing, diagnosing, sensing, selective protection, targeted removing, measuring, and assisting medical treatment functions at cell structure level or micro-organ (up to 500 micron scale) level for medical purposes including cancer and treatment of blood related diseases.

48. The method of using a fabricated micro-device in claim 47, using one or more micro-devices for cancer treatment comprising the steps of: (a) selectively attaching micro-devices with drug delivery functions onto cancer cells; (b) triggering injection function in the micro-devices; and, (c) injecting drug into cancer cells.

49. The method of using a fabricated micro-device in claim 47, said method comprising the steps of: (a) selectively attaching micro-devices with high optical reflectivity onto healthy cells; (b) carrying out laser treatment to destroy unhealthy cells; and, (c) removing unhealthy cells in the treatment due to exposure to the laser.

50. The method of using a fabricated micro-device in claim 47, said method for diagnosing, sensing, and measuring functions comprising the steps of: (a) delivering one or more micro-devices to a targeted measuring site; (b) said micro-device having properties selected from the group comprising of: signal sensing unit, memory unit, logic processing unit, signal transmitter, and micro-surgery; (c) performing measurement on the targeted site; (d) recording data in memory unit; (e) optionally triggering operations using the logic processing function; (f) optionally, carrying out surgery using the said micro-device; (g) retrieving the micro-device or micro-devices; and, (h) analyzing the recorded data.

51. The method of using a fabricated micro-device in claim 47, for diagnosing, sensing, and measuring functions comprising the steps of: (a) delivering one or more micro-devices to a targeted measuring site, (b) said micro-device having properties selected from the group comprising of: signal sensing unit, memory unit, signal transmitter, logic unit for on-site decision making and micro-surgery; (c) performing measurements on the targeted site; (d) recording data in memory unit; (e) analyzing the data performed by said micro-device; (f) deciding the course and type of micro-operations based on data analysis and pre-programmed logic decisions by said micro-device; and (g) performing micro-operations on the measured site.

52. The method of using a fabricated micro-device in claim 47, said method for cancer cell detection comprising the steps of: (a) selecting at least one micro-device(s) having at least one electrical property measurement unit; (b) delivering said micro-device to a measurement site; (c) measuring at least one or combination of properties selected from the following group comprising: surface charge, charge density, resting potential, electrical potential, electro-chemical potential, surface current, bulk current, and current density is measured at the measurement site.

53. The method of using a fabricated micro-device in claim 52, selecting a micro-device for sensing comprising at least one voltage comparator.

54. The method of using a fabricated micro-device in claim 53, whereby the said voltage comparator has a preferred voltage measurement sensitivity better than 5 mV.

55. The method of using a fabricated micro-device in claim 47, said method is used for cancer cell detection comprising the steps of: (a) delivering micro-device(s) to the site of measurement; and, (b) measuring at least one or combination of the following parameters at the measurement site selected from the group comprising of: surface charge, resting potential, electro-chemical potential, electrical potential, surface current, bulk current, surface wettability, contact angle, adhesion properties, temperature, density, friction, hardness, surface tension, trace chemical concentration, pH, liquid flow rate, pressure, optical properties, absorption, adsorption, and composition.
